(19) United States
(12) Patent Application Publication
(10) Pub. No.: US 2015/0065901 A1
(43) Pub. Date: Mar. 5, 2015

(54) UNIVERSAL BREATH SAMPLING AND ANALYSIS DEVICE

(71) Applicant: Capnia, Inc., Redwood City, CA (US)

(72) Inventors: Anish BHATNAGAR, Redwood City, CA (US); Anthony D. WONDKA, San Ramon, CA (US)

(21) Appl. No.: 14/473,878

(22) Filed: Aug. 29, 2014

Related U.S. Application Data
(60) Provisional application No. 61/872,514, filed on Aug. 30, 2013.
(60) Provisional application No. 61/872,450, filed on Aug. 30, 2013.

(51) Int. Cl. A61B 5/097; A61B 5/08

(52) U.S. Cl. CPC A61B 5/097; A61B 5/082
USPC 600/532; 600/543

(57) ABSTRACT

A breath analysis device is described which obtains a desired segment of one or more breaths, and analyzes that or those samples for compositional analysis. A pneumatic control system may obtain these segments homogeneously, may reduce the amount of gases included from other segments of the breath, and may reduce mixing with other segments once obtained. These pneumatic control systems can be used for on-board compositional analysis, or for modular or off-board compositional analysis.

### CROSS-REFERENCE TO RELATED APPLICATION

This application claims the benefit of U.S. Provisional Application Nos. 61/872,514 and 61/872,450, both filed Aug. 30, 2013, the contents of both of which are incorporated herein in their entireties.

### FIELD OF THE INVENTION

The present disclosure relates to the field of breath analysis for monitoring, diagnosing and assessing medical conditions by measuring markers in the breath.

### BACKGROUND

Some breath analysis devices acquire a breath sample using a controlled breath hold and forced exhalation maneuver by the patient. Other breath analysis devices acquire the breath sample from the patient by applying a vacuum sampling tube coupled to the patient's expiratory flow. The latter technique, which has several advantages, is described in the present disclosure. In this type of sampling device, the target analyte will typically be in a certain segment of the patient's exhaled breath, for example the beginning, middle or end of the exhaled breath. These different segments correspond to the physiologic origin of the analyte, for example alimentary, airways, deep lung, or systemic. In some prior art described by Natus (U.S. Pat. No. 6,544,190), end-tidal CO gas level was reported by measuring the all the sections of the exhaled gas over several breaths, then applying a transfer function to correlate the measurement to an end-tidal value. It is believed this technique had several limitations, such as potential inaccuracy because of the transfer functions not being able to accommodate the wide variety of clinical situations one will likely encounter.

The present disclosure contemplates novel pneumatic control systems, which are intended to prevent mixing of the targeted breath section with other sections. In addition the present disclosure describes applying these novel control systems to both on-board analysis, off board analysis and modular analysis, as will be described in the forgoing. Finally, the present disclosure also describes both single breath and multiple breath analyses as opposed to only single breath analysis, and analysis of other sections of the breathing pattern besides only the deep lung or end-tidal section analysis.

### BRIEF DESCRIPTION OF THE FIGURES

FIG. 1 is a pneumatic schematic of a prior art system for collecting and measuring a breath analyte sample.

FIG. 2 is a pneumatic schematic of a system of an embodiment which measures a breath sample without suspending the movement of the sample through the system.

FIG. 3 shows a timing diagram of the system shown in FIG. 2, showing the valve control during a test sequence including selecting a breath, shunting the end-tidal section of the selected breath to a sensor, and measuring the breath sample for an analyte.

FIG. 4 is a pneumatic schematic showing a removable and replaceable cartridge which receives the gas sample that is intended for analysis.

FIG. 5 is a pneumatic schematic showing a point of care breath sample collection and sample segment isolation instrument which is connectable to an off-board breath analyte sensor for analyte analysis.

FIG. 6 is a flow diagram describing a sequence of operation of the system.

FIG. 7 is a flow diagram describing operation of the system described in FIG. 6 with user selectable options related to the test being conducted.

FIG. 8 is a pneumatic schematic describing an alternative to the pneumatic system described in FIG. 2 in which the pump direction is reversed to divert the sample intended for measurement to the sensor.

FIG. 9 is a pneumatic schematic describing an alternative pneumatic system for obtaining a sample of a section of a breath in which the sample after collection is pushed into a removable chamber for off-board analysis.

FIG. 10 is a pneumatic schematic describing an alternative pneumatic system for obtaining a sample of a section of a breath in which the sample is drawn through a removable chamber for off-board analysis.

FIG. 11 is a pneumatic schematic describing a pneumatic system for obtaining a sample of a section of breath in which patient gas is drawn through a bypass tube until a desired section of a desired breath is identified which is then diverted into a sample isolation chamber.

FIG. 12 graphically describes breath sensor signals measuring the gas of one breath, using the example of CO2 measuring in the upper graph and breathing airway pressure in the lower graph, and shows the breath cycles and gas sections related to the different breath cycles,

FIG. 13 is a pneumatic schematic describing a pneumatic system for obtaining a sample of a section of breath showing the different sections of a breaths traveling through the system and in which includes a vent port coupled with the inlet of a sample trap to purge gas prior to trapping the analyte for analysis

FIG. 14 graphically shows as a function of time a series of breaths corresponding to the breaths and breath sections of gas shown in FIG. 13.

FIG. 15 shows a cross-sectional detailed side view of a removable analyte trap, such as shown in FIGS. 4 and 10, to facilitate offboard analysis of the analyte.

FIG. 16 shows the trap shown in FIG. 15 with a desired section of gas from a desired breath filling the trap, with the inlet valve closed to isolate the sample.

FIG. 17 schematically shows a sample transfer module including a syringe-type device to obtain the sample from the system shown in FIG. 3.

FIG. 18 schematically shows an option to the system shown in FIG. 13 in which multiple sample traps are included to broaden the utility of the system.

FIG. 19 shows a pneumatic diagram of a passive sample collection apparatus for collecting an end-tidal section of a breath, which can be coupled to a subject's respiratory cycle.

FIG. 20 shows the apparatus of FIG. 19 during the inspiratory state of the subject.

FIG. 21 shows the apparatus of FIG. 19 during the expiratory state of the subject.

FIG. 22 shows a means of withdrawal of the end-tidal sample shown in FIG. 19, by removal of the sample through a port in the expiratory limb of the apparatus.

FIG. 23 shows an optional means of withdrawal of the end-tidal sample show in FIG. 19 by removal of the expiratory limb of the apparatus.

FIG. 24 graphically shows as a function of time a subject's breathing cycle over a series of breaths.

FIG. 25 graphically shows a detailed view of one of the breaths shown in FIG. 24.

FIG. 26 shows the apparatus of FIG. 19 at a time that the breath from FIG. 25 occupies the apparatus.

FIG. 27 shows an alternative to the apparatus of FIG. 19 showing an adjustable volume expiratory limb of the apparatus so as to adjust the sample collection volume of the expiratory limb based on the subject's size and the test being performed.

FIG. 28 graphically shows the breath from FIG. 25 in which the end of exhalation of the breath is segmented graphically into 4 sections, these sections optionally corresponding to the volume adjustment setting on the expiratory limb of the apparatus shown in FIG. 27.

FIG. 29 shows an automated version of the apparatus shown in FIG. 19 automated for identifying and collecting an end-tidal section of gas from a desired breath, shown during an expiratory cycle and shown exhausting the gas from a breath identified as a breath not suitable for analysis. Such a device can be used to verify an appropriate breath is sampled, and can prevent a subject from trying to fool the device.

FIG. 30 shows the apparatus of FIG. 29 during an expiratory cycle in which a breath is identified as being suitable for analysis, showing the end-tidal section of gas passing through the expiratory limb sample collection container.

FIG. 31 graphically shows a breath parameter of a series of breaths as a function of time, showing breath 18 being identified as a breath suitable for analysis by the apparatus shown in FIGS. 29 and 30.

FIG. 32 is a pneumatic schematic similar to the apparatus of FIG. 27 combining the features of automation shown in FIGS. 29 and 30 and adjustment of the sample collection compartment to match the expected sample volume, the adjustment performed manually, automatically or semi-automatically, the volume adjustment optionally based on the measured breathing pattern shown in FIG. 31.

### DETAILED DESCRIPTION

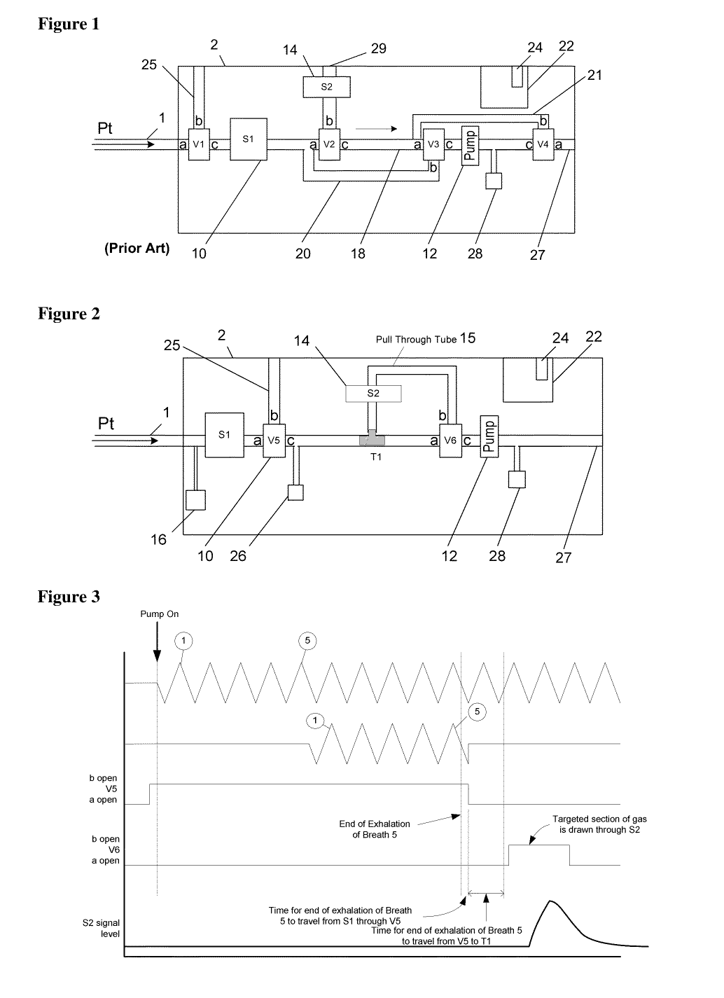

FIG. 1 depicts a prior art device which includes an inlet for attachment of a sampling cannula 1, and an instrument 2. The instrument includes an inlet connector for cannula attachment, an inlet value V1 to switch between ambient 25 and patient gas Pt, a breathing pattern sensor S1 to query the breathing pattern, a sample tube 18 to contain the sample which is to be analyzed, an inlet and outlet valve, V2 and V3, to the sample tube, a bypass tube 20 to divert other gases around the gas sample in the sample tube, a push tube 21 to push the gas in the sample tube to the gas composition sensor S2, a pump to draw the sample from the patient and to push the sample to the gas composition sensor, a valve V4 to control whether the pump is drawing from the patient or pushing the sample to the gas composition sensor.

FIG. 2 describes an embodiment. The pneumatic control and sampling system can be performed with as little as two 3 way valves rather than three or four, which minimizes the cost and complexity of the overall apparatus. In addition, positioning of the section of the breath sample may be precisely determined since the response time tolerances of the least number of valves need to be accounted for. Also, the targeted sample can be analyzed by the sensor S2 without stopping it somewhere in the system. Keeping the sample in motion and minimizing the time between when the sample exits the patient and when it is analyzed, may minimize the chance of mixing of the sample with gas from other sections of the breath. In this configuration, gas is drawn from the patient through S1, V5, T1, V6 and the pump. When a desired section of breath from a desired breath is identified by S1, at the appropriate times, V5 and V6 are switched from ports a to ports b, and without interrupting gas flow, the targeted sample is diverted to and through the composition sensor S2 by being pulled through V6 by the pump. As it travels into and/or through the Sensor S2 the sample is analyzed for the analyte(s) in question. The junction T1 that bifurcates the patient flow path with the sample analysis path can be a Tee or can be a valve for further fidelity of the system. If a Tee, one way check valves can be placed before or after the Tee to prevent entrainment of unwanted gases and unwanted mixing. Calibration of the system follows the same approach using a known level of analyte. The system 2 includes the patient inlet Pt, a cannula 1 or collection circuit, an ambient inlet 25, an analyte sensor S2 or 14, a sensor pull through tube 15, a control system 24, a user interface 22, optionally a patient inlet sensor 16 such as a pressure transducer, a breathing pattern sensor S1, an inlet control valve V5, a flow path sensor 26 such as a pressure transducer, a tee T1, a flow path selector valve V6, a pump P, a second flow path sensor 28 such as a pressure transducer, and an exhaust 27.

A closer description of how the system operates is shown in FIG. 3, which describes the breathing pattern signal measured at S1 and the control of the valves V5 and V6, and the response of the analyte sensor S2 to the sample. In the example shown, an end-tidal sample is being targeted for analysis, however the same principle applies to other sections of the breath. As shown in the example, when the end-of-exhalation of the breath being targeted is identified by S1, a time counter is started. In the example shown, end-of-exhalation is identified by the breathing parameter signal crossing zero from a positive value, such as would be the case with a pressure or flow sensor. Other times of sensors can be used such as thermal sensors, capnometers and others, in which case the end-of-exhalation may be identified by a different characteristic in the signal, such as a change in direction, the derivative crossing zero and other such characteristics. Regardless of the sensor type and signal characteristic, it is known that it will take X seconds for this point of the breathing pattern (the end of exhalation) to travel from the exit point of S1 to the middle port or port c of valve V5, based on flow rate and tubing dimensions. When this point of the breath reaches that point, valve V5 switches so that gas from the patient is no longer drawn into the device. The valve may be controlled to switch slightly prematurely to assure that no patient gas after the end of exhalation reaches V5. Then, when the end of exhalation reaches the mid port of the tee T1, valve V6 is switched to divert the flow of the targeted sample to the analyte sensor S2. There may be deliberately a slight delay in the switching of V6 to assure that no gas before the sample being targeted is inadvertently rerouted to S2. The targeted sample is then pulled through S2 for an appropriate and precisely controlled duration, after which V6 is switched again and gas flow through S2 ceases. During the time that the gas is pulled through the sensor, at first the ambient gas in the tubing leading to the sensor is pulled through, to which the sensor minimally reacts, and at a time after switching of V6 the beginning of the sample in question enters S1, and at a known time after switching of V6 the end of the sample reaches the sensor. V6 can be controlled to switch again exactly at that time, or a time before or after that time, but always in a predetermined manner that matches the calibration procedure. When the sample itself enters S1, the sensor begins to react to the analyte, and this signal response is measured in the appropriate manner, for example integration, and then correlated to a quantitative measurement of the analyte, based on the calibration factors established earlier.

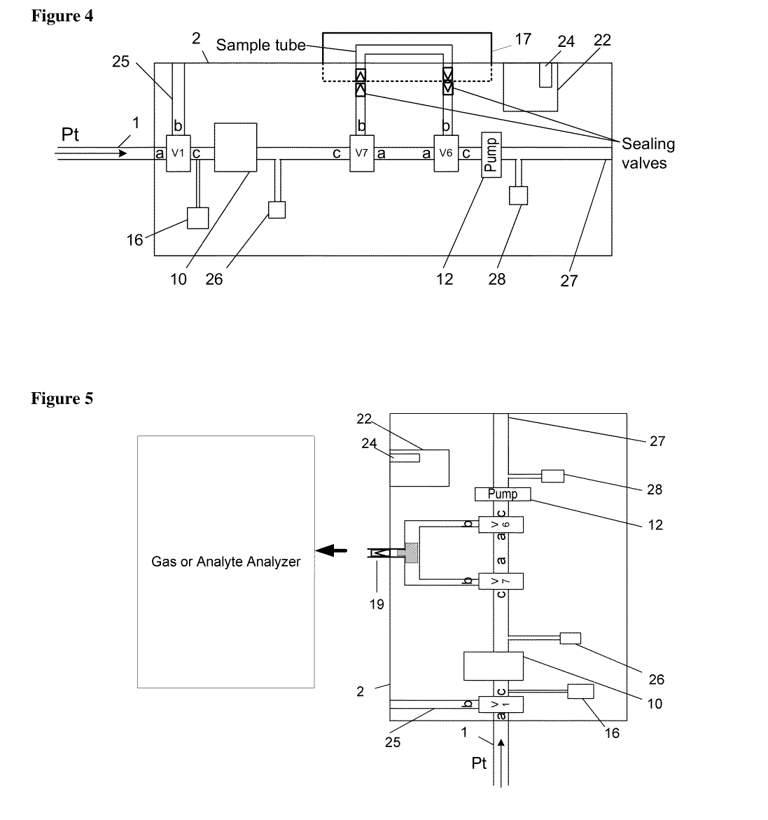

FIG. 4 shows some variations of the systems shown in FIGS. 2 and 3. In this system the tee T1 is replaced by a 3 way valve V7, to provide more precise control of the gases flowing into and out of T1 in the previous example, for example to prevent inertia related mixing of gases from different breath sections. In addition, FIG. 4 shows a removable sample collection device 17, which can be used to bring the sample to an off-board analyzer. The sample is preserved typically in a tube, canister, cylinder or syringe, and protected from contamination from outside gases with a series of one-way check valves. Now that the sample is preserved in this collection device 17, it is no longer prone to mixing with patient gases from other breath sections, and the fact that it is static is of no concern. The sample can be then drawn out in aliquots or in its entirety and injected into the desired analyzer or instrument(s), or the sample compartment can be remove-ably designed to conveniently attach to an analyzer or instrument for convenient injection or uptake into the instrument. The sample can also be stored indefinitely for future analysis. Alternatively as shown in FIG. 5, the entire breath collection instrument itself can be modularly designed and of the correct form factor to connect to the composition analyzer via a analyzer connection 19, which may be at a central location. In this example the apparatus is typically a miniature hand-held device. For example, the collection can be taken in the field, or in an ambulance, at home, at a screening clinic, in a village, and later when reaching a facility, the instrument can be delivered to the laboratory and connected to the composition analyzer.

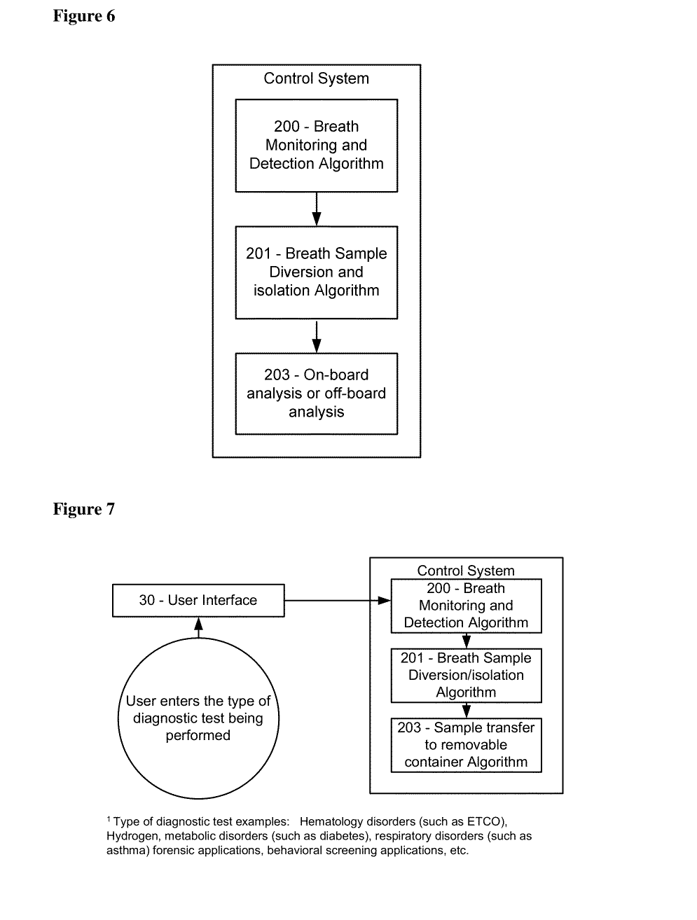

In FIG. 6 the basic steps of the procedure are shown. Step 1: breath monitoring and detection, in order to identify an appropriate breath, and the appropriate section of gas within that breath, using the sampling system and tubing, and appropriate sensor(s) and algorithms; Step 2: the appropriate sample is diverted and isolated from other breath gases, which is accomplished by special control systems, pumping, valves, tees and tubing with associated algorithms; Step 3: On-board analysis and/or preservation and transfer to an off-board analyzer.

FIG. 7 describes the universality of the system, with a user selection to allow the user to specify the type of analysis to be performed. The specific analysis selected will automatically enable the appropriate control systems and algorithms to work accordingly. For example an end-tidal sample can be sampled, or multiple breaths can be sampled, or a breath of a certain breath profile can be sampled, all of which are optimized for the diagnostic test being selected by the user and performed. Test can be for hematology disorders such as ETCO measurements for hemolysis, alimentary disorders such as hydrogen measurements, metabolic disorders such as diabetes, respiratory disorders such as asthma, forensic applications and behavioral screening applications, etc.

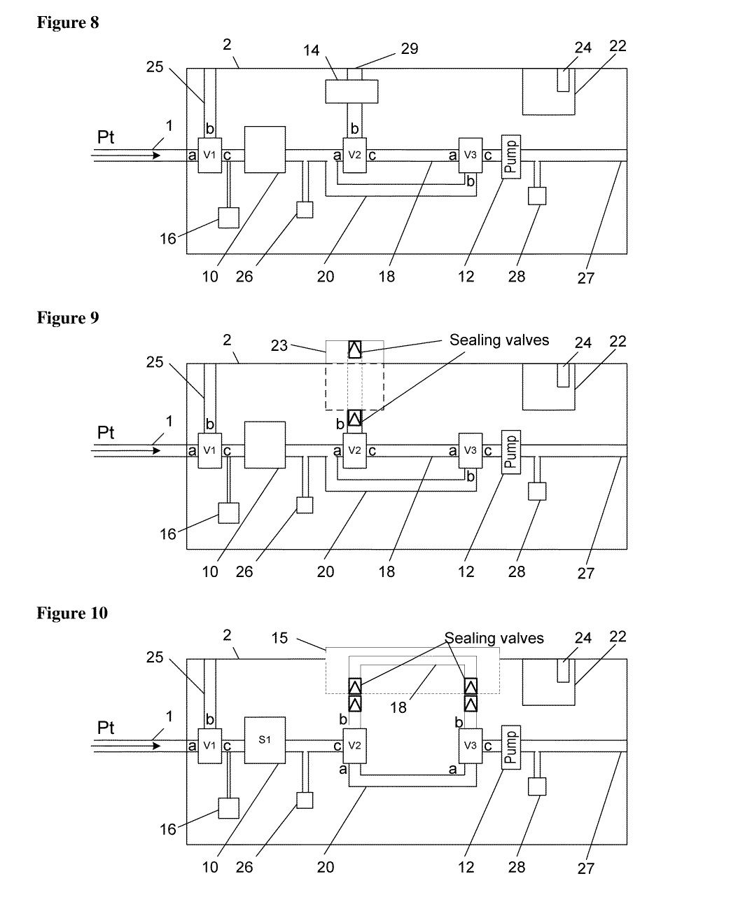

FIG. 8 describes an alternative pneumatic control system in which the sample of interested is isolated in the tube 18 between V2 and V3, after which the Valve V2 changes from port a open to port b open and the pump direction is reversed and the sample is pushed to the sensor 14.

FIG. 9 describes a variation of the system in FIG. 8 in which the sample is sent to a removable collection container 23 for off-board analysis. The sample is protected in the container 23 by check valves, self-sealing ports, or the like.

FIG. 10 describes an alternate pneumatic control system in which the unwanted gas is routed between V2 port a and V3 port a, and in which the wanted gas is routed between ports b of V2 and V3 and placed in a sample tube 18. The wanted gas sample can be analyzed on-board or off-board as previously described.

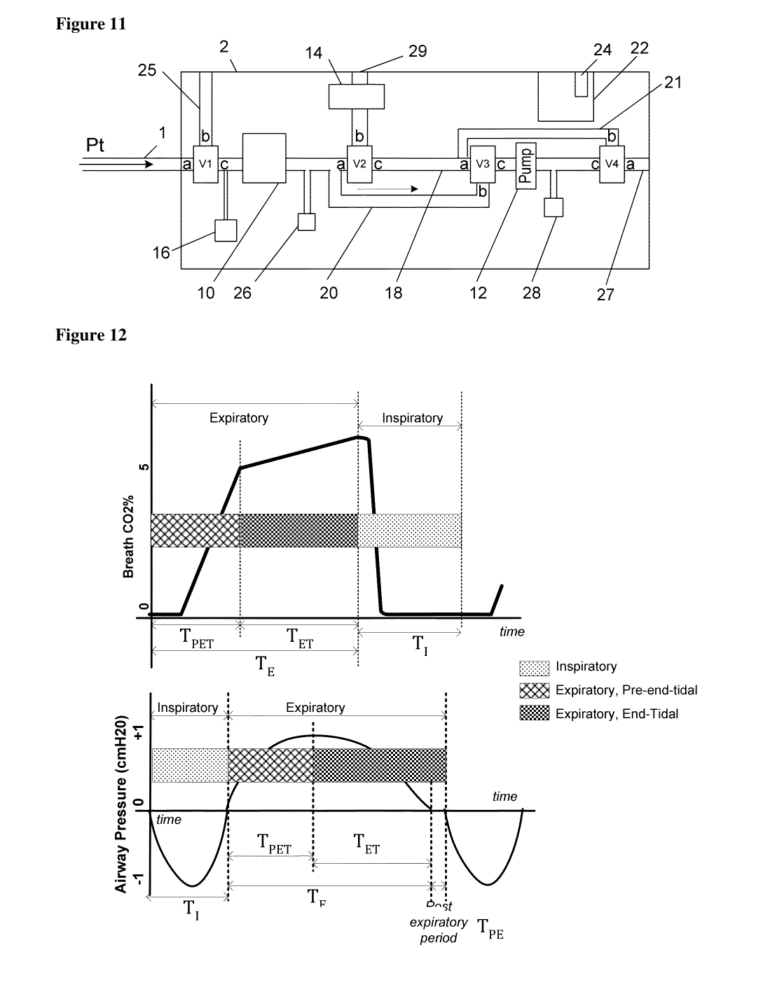

FIG. 11 describes an alternative pneumatic control system in which the patient gas is diverted around the tube 18 through tube 20, between V2 port c and V3 port a, until a desired section of gas is identified by the sensor S1. When this desired section reaches V2, the appropriate valve switching takes place and routes the desired sample into the tube 18 between V2 port c and V3 port a.

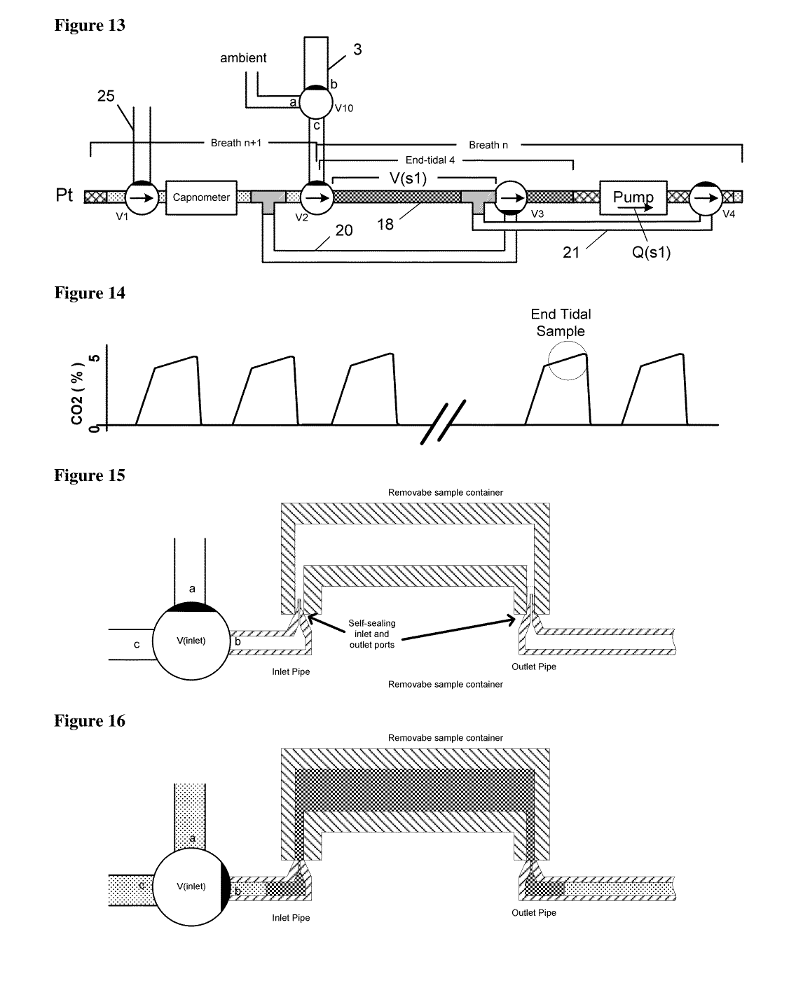

FIG. 13 describes a variant of the system of FIG. 11 in which there is a Valve V10 which acts as a vent to purge any unwanted gases between V2 and V10, such that the resultant sample ultimately placed in the collection device 3 is not diluted or contaminated with other gases. FIG. 12 describes a typical breath curve based on capnometry and airway pressure, and shows the different sections of gas within a breath period that are being drawn through the apparatus shown in FIG. 13. In FIG. 12, T(PET) is pre-end-tidal time; T(ET) is end-tidal time; T(I) is inspiratory time; T(E) is expiratory time; T(PE) is post-expiratory period. The upper graph indicates a typical breathing curve based on a capnometry signal, and the lower graph indicates a typical breathing curve based on breathing pressure. The main different sections of breath gas are depicted schematically in the graphs accordingly, corresponding to the gas sections in FIG. 13. FIG. 14 describes a series of breaths on a time scale as depicted by a capnometry signal, and shows the breath, breath n, being targeted in this series of breaths for the example shown in FIG. 13.

FIG. 15 describes a sample container of the system shown in FIGS. 4 and 10 in which the sample container is attached to the collection device with remove-ably attachable self-sealing connectors, so that the container can be freely removed without contamination of the sample. FIG. 16 shows the sample container of FIG. 15 filled with the desired sample, in this example, the end-tidal gas from breath n from FIG. 14. The types of containers can be for example a tube with sealing or self-sealing inlets and outlets, a gas tight syringe with an inlet only, a tube which first is evacuated with a self-sealing inlet and which draws the sample inward optionally via its internal vacuum, a tube which is inserted in place of the sample tube 18 with a sealing or self-sealing inlet and outlet, a tube or compartment with a valve on one end.

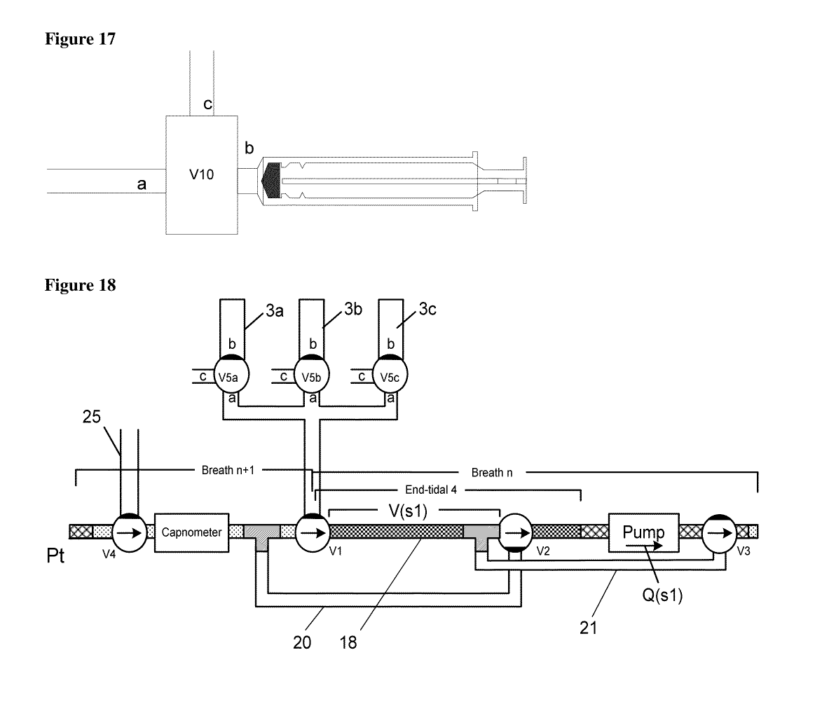

FIG. 17 shows an alternative to FIG. 13 in which the sample is drawn into a syringe or similar device such as a cuvette or pipette, for off-board analysis. In this manner, multiple syringes can be filled and labeled accordingly, for a fill work up on the patient. This embodiment can be used in conjunction with the user-settable input described in FIG. 7. FIG. 18 shows a variant of the system of FIG. 13 in which there are multiple valves and collection containers to collect and analyze multiple samples.

The system described in FIGS. 1-18 can be useful for collecting and measuring end-tidal gas samples, as well as samples from other sections of the breath. It can be used for measuring for example CO in the breath, or other gases, such as H2, NO, and others. It can be used for measuring other non-gaseous substances in the breath as well as gaseous markers. The compositional analysis and breath pattern sensing can be two different sensors, or one sensor. The system can be used to collect and measure an analyte in the end-tidal section of a breath, or other sections of the expiratory cycle such as for example the middle airways. A host of clinical syndromes can be assess or diagnosed using this system.

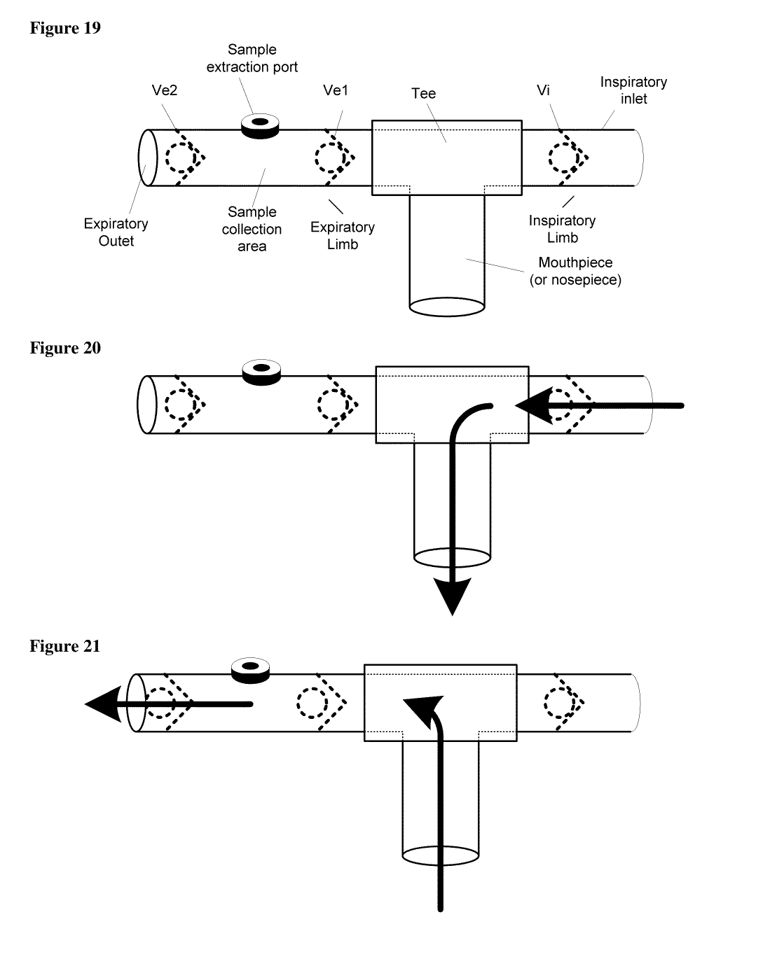

FIGS. 19-32 describe an optional apparatus and method in which the a breath sample is collected passively when coupled to the subject's respiration pathway, such as coupled to the mouth,

There are two techniques described in the prior art for obtaining an end-tidal breath sample for analysis of the alveolar gas. Some Breath analysis devices acquire a breath sample using a controlled breath hold and forced exhalation maneuver by the patient into a collection device. Other breath analysis devices acquire the breath sample from the patient by applying a vacuum to a sampling tube that is in communication with the patient's expiratory flow. Both of these techniques have limitations. In the case of a breath hold maneuver, the breath hold itself may alter the concentrations levels of the gases in the lung, and therefore may provide an inaccurate understanding of the underlying condition that is being evaluated. Further, the maneuver needs to assure that homogenous end-tidal gas is collected, and that the patient for example doesn't breath in their nose while pausing to press their lips against the collection device half way into exhalation. In addition, a test subject or patient may not properly follow the maneuver instructions, or there could be variability from test to test because of not strictly adhering to the instructions. Or, if performing back to back maneuvers to collect a sample, there is no way of knowing when the gas concentrations in the patient's lung reach respiratory equilibrium and are ready for a test.

In the case of collection via a vacuum and sampling tube, this technique has been demonstrated to be reliable and accurate, however, may not be optimized for field deployment.

In FIGS. 19-32 a sampling device is described that obviates the need for and related drawbacks of a breath hold maneuver. In addition, some embodiments collect a relatively large sample of end-tidal gas, and can be employed with minimal costs and maximum reliability, on both alert and non-alert patients, and on patients of all ages. Some embodiments further allow for flexibility in the sample collection, based on the intended use and clinical application, such as configurable sample collection volumes, sample collection from different sections of the breathing curve, and verification sampling only breaths that are representative of the breath type that should be sampled for the particular clinical application. The embodiments can be designed as a passive system not requiring mechanical parts only for maximum simplicity, or can include some electro-mechanical parts and a control system for added intelligence when used in more exacting clinical applications.

FIG. 19 describes an embodiment of the system. A novel breath pass-through apparatus is shown. The user applies the mouthpiece to their mouth and simply breathes normally. Inspired air travels in through the inspiratory inlet unabated, through the one-way inspiratory check valve Vi in the inspiratory limb, and into the respiratory tract via the mouthpiece, as is shown in FIG. 20. Exhaled air travels out of the respiratory tract, through the mouthpiece, through the one-way expiratory check valve Ve1 in the expiratory limb, and out of the apparatus through the one-way expiratory check valve Ve2, as is shown in FIG. 21. The user breathes normally and naturally, and the apparatus does not inherently change the breathing mechanics. A nose clip can be applied to the nose to assure that all of the breathing is through the mouth. At any given time the apparatus can be withdrawn from the mouth, and by definition, expiratory gas must reside in the sample collection area between Ve1 and Ve2, as long as the user has breathed one or more breaths with the apparatus in place. The apparatus is typically designed so that the gas pathways are as small as possible without adding breathing resistance, so that the apparatus does not alter the breathing mechanics and respiratory equilibrium. This can be done with gas pathway diameters of about ⅜″ to ¾″ without any noticeable breathing resistance. The different sections in the apparatus are designed with minimal volumes between Vi and the Tee, in the mouthpiece, and between the Tee and Ve1, to avoid unnecessary dead-space and in order to place the gas from the very end of exhalation between Ve1 and Ve2. The sample can be extracted for analysis through the extraction port. The apparatus is versatile and can be used differently depending on the clinical application. For example, the patient can breathe “normally” in order to collect a gas sample from a normal tidal volume breath. Or, the patient can breathe “deeply”, to collect an expiratory reserve volume gas sample. While the apparatus is shown with a mouthpiece patient interface in this application, other respiratory track interfaces can be used such as a nasal mask, nasal pillows, nasal cannula, face mask, tracheal tube, bronchial tube, bronchoscope, or other interfaces. While the example is shown during spontaneous ventilation of the subject, with little or no modifications the system can be used by coupling to a mechanically ventilated subject, such as by attachment to the breathing circuit.

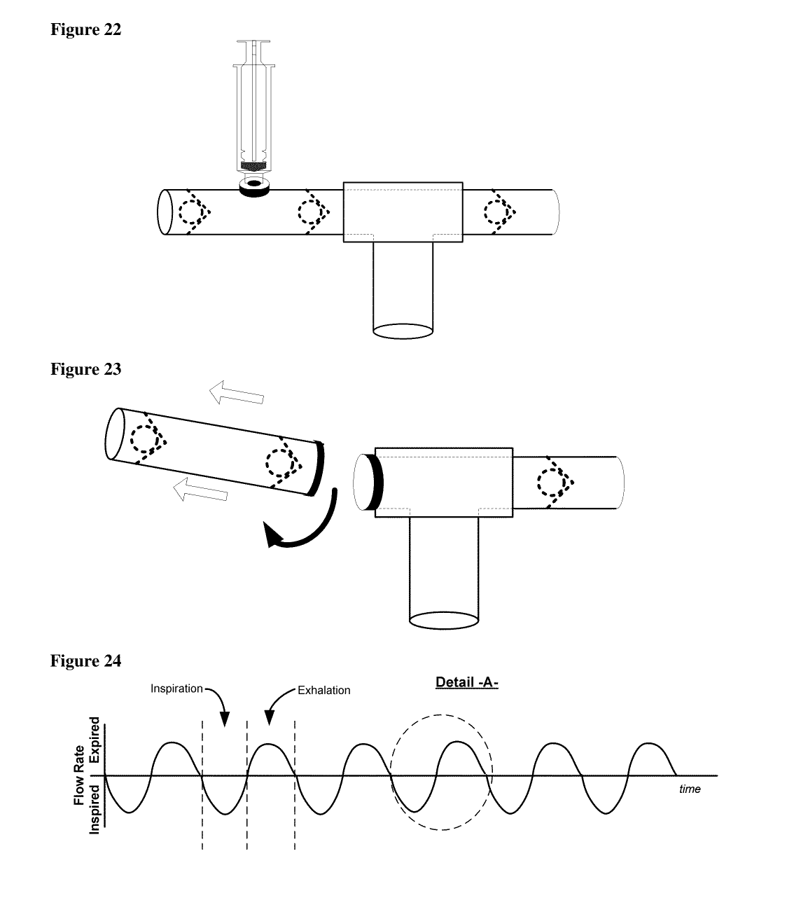

FIG. 22 shows an example of how the sample can be extracted from the expiratory limb for analysis, for example using a syringe type device attached to a self-sealing port, and drawing the sample into the syringe where it is preserved until the analysis is performed. In some point of collection and field applications, the syringe may include a sensor media, for example a paper or plastic with the proper chemistry, which is altered for example in color when exposed to the analyte that the patient is being test for. FIG. 23 shows an alternative way to transfer the sample to an instrument for compositional analysis, by removing the sample collection area from the expiratory limb of the apparatus. Multiple samples can be taken from the same patient if required by the situation.

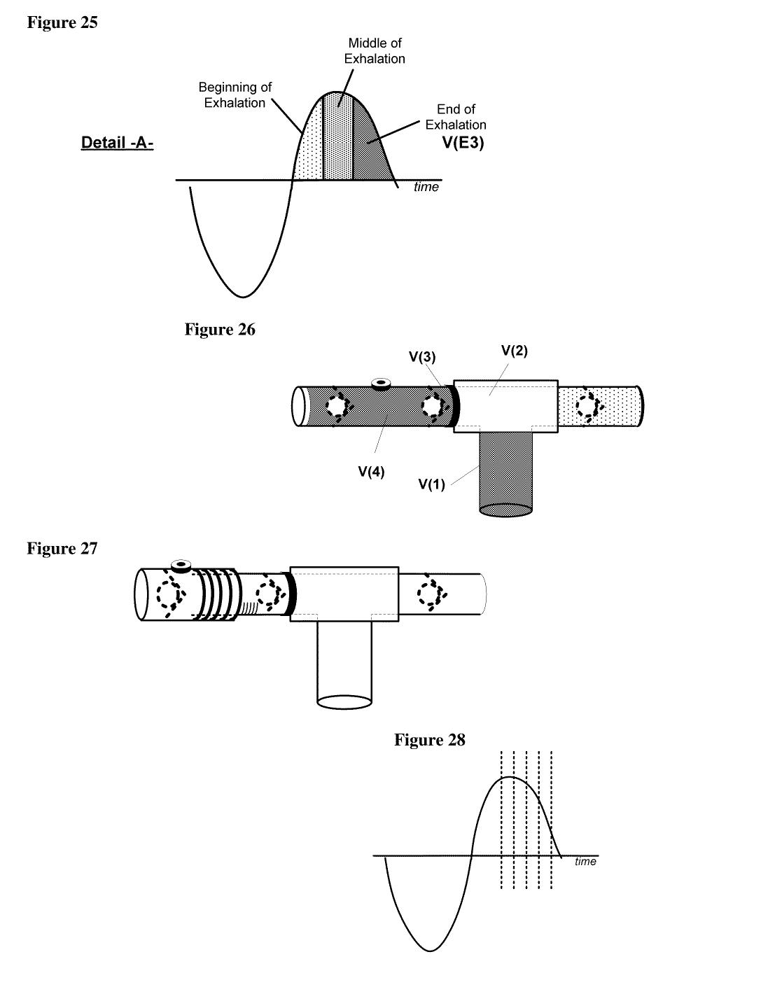

The apparatus described in FIGS. 19-23 can be designed to collect a gas sample from a certain section of the expiration cycle. In FIG. 24 a typical breathing curve is shown as a function of time based on airway flow measurements, with an inspiratory section of the curve and an expiratory section of the curve. FIG. 25 is a more detailed view of a curve of a typical breath from FIG. 24, graphically showing that the expiratory section of the breathing curve can be broken down into multiple different sections. In the example shown it is divided into three sections, beginning, middle and end, although exhalation can be divided into more or less sections. Each section has the potential to contain a different mixture of gas concentrations. In one embodiment, the end-tidal section or final third section of exhalation is desired to be collected for measurement, from a normal tidal volume breath. This amount of volume from the patient is represented by the area under the flow curve, or V(E3) in FIG. 25. In this case it is important that the additive volumes of the sections of the apparatus shown in FIG. 26, sections V(1), V(2), V(3) and V(4), be less than the volume V(E3), in order to assure that V(4) in FIG. 26 contains only gas from the end-of-exhalation. For example, exhalation may be 500 ml, and the final third of exhalation may be 150 ml, and V(1) may be 15 ml, V(2) may be 20 ml, V(3) may be 5 ml, and V(4) may be 75 ml, giving the apparatus a 30% safety factor in assurance that the collected sample will be a pure sample from the targeted section.

There may be a need for some flexibility of the system, for example testing different sized patients and therefore different V(E3)'s ranging from 5 ml to 750 ml. Or, for example, the test may require obtaining more or less precise sections of gas from the expiratory cycle. In some cases this is handled by different sized collection apparatus. In other cases this requirement in collection volume ranges can be handled by an adjustable apparatus, to adjust to the volume of V(E3). As shown in FIG. 27, the sample collection area volume in the expiratory limb can be adjusted and increased or decreased depending on the expected V(E3) volume. The adjustment can be accomplished by a replaceable section, or by a moveable section, for example with threads or a sealing slide, or by a module expiratory limb that can be switched with different sized modules. In the latter case, the apparatus may be provided as part of a kit, with different sized expiratory limbs indicated for different test requirements. In addition, the sample collection area can include graduated markings to indicate to the user the volume to which the apparatus is adjusted or set. Alternatively, or in addition, the apparatus can be adjustable for the purpose of collecting a gas sample from a different percentage of the end-of exhalation. For example, as shown in FIG. 28, the second half of exhalation can be divided into four or five segments, and the adjustment scale on the apparatus shown in FIG. 27 can correspond to each of these segments. The finer the setting of the volume of the expiratory limb in FIG. 27, the more precise the collection of gas from the expiratory cycle shown in FIG. 28 can be.

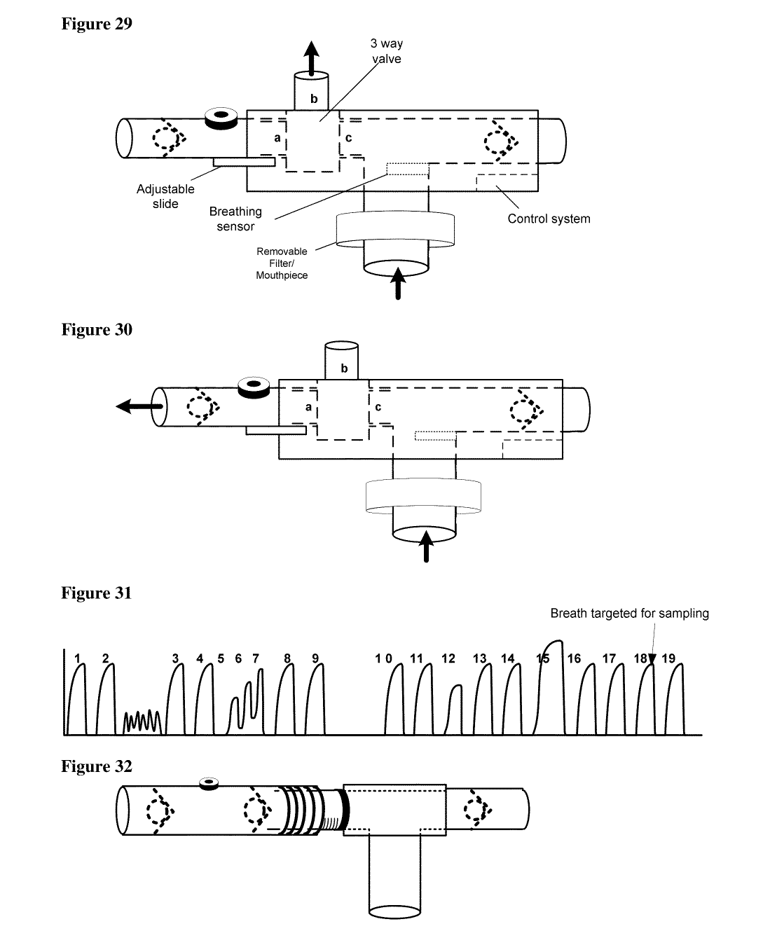

In some cases, it is desired or needed to add some control sophistication to the apparatus, in order to automatically assure that an appropriate sample from an appropriate breath is appropriately captured. In this embodiment, shown in FIG. 29, the one-way expiratory valve Ve1 of FIG. 19 is replaced with an electronically controlled 3 way solenoid valve. When the patient breathes through the apparatus, breaths that are not desired to be sampled are expired out through port b of the 3 way valve as shown in FIG. 29, and a breath that is desired to be sampled is expired out through port a of the 3 way valve as shown in FIG. 30. A breathing sensor is placed in the breathing gas flow path to measure the breathing pattern so that breaths can be classified as appropriate or inappropriate, based on thresholds, criteria, and algorithms. This information from the breathing sensor is used by a control system to control the 3 way valve accordingly, by routing certain breaths through port b and others through port a, as desired. The breathing sensor can be for example a flow sensor, temperature sensor, pressure sensor, or gas composition sensor. Since the apparatus is of some complexity and cost, the mouthpiece can be disposable and the balance reusable, in which case the mouthpiece includes a two way bacterial filter to prevent cross contamination between users. A simple flush kit and procedure can be used in between patients to remove any residual patient gases from the previous patient, to avoid sample contamination of the next patient. In FIG. 31, the breathing parameter signal from the breathing sensor of FIGS. 29 and 30 is plotted as a function of time for a series of breaths. Algorithms in the apparatus' control system determine which breaths are rejected for sampling, and which breath is targeted, in this case breath 18. The 3 way valve can be switched to port a after breath 17 is expelled out of port b for example, then breath 18 is expelled through port a and into the sample collection area, then the valve is switched again to port b, preserving the end-tidal sample from breath 18 in the sample collection area, and preventing contamination from other breaths. After breath 18 is complete however, the control system by using the information from the sensor, confirms that breath 18 was still an appropriate breath to sample. If this is confirmed affirmatively, then the sample collection is completed and the user can remove the apparatus at any time, otherwise if it is decided that the sample was in-appropriate after all, then the process of finding an appropriate breath is repeated and eventually the sample from breath 18 in the sample collection area is displaced with a sample from the next targeted breath. In an additional embodiment, the control system in conjunction with the breath sensor and 3 way valve, can be used to collect the end-tidal section of multiple breaths in the sample collection area, by the proper switching and timing of the 3 way valve.

In some cases, it may be important to obtain a sample from a certain type of breath. For example, after a sigh breath, or a breath after some other type of breath or during or after a certain type of breathing pattern chosen for the diagnostic test at hand. In these cases, the control system and the appropriate algorithms are used to capture the appropriate sample. A user interface may be included which allows the user to enter a certain sampling protocol, and the system then automatically makes the necessary adjustment and algorithm changes in order to conduct the desired test. The system can also be adaptive and automatically or semi-automatically adapt to the prevailing clinical situation and conditions. The specific analysis selected will automatically enable the appropriate control systems and algorithms to work accordingly. For example an end-tidal sample can be sampled, or multiple breaths can be sampled, or a breath of a certain breath profile can be sampled, all of which are optimized for the diagnostic test being performed. Adjustments to the expiratory limb can allow the sample collection area to collect different portions of gas from the expiratory cycle, for example a section of gas from the middle airways rather than an end-tidal section as described in previous embodiments. The position of valves in the expiratory limb, together with the breath rate and breathing volumes being measured by the breath sensor, can dictate what area of the expiratory gas is isolated between the valves for analysis.

In FIG. 32 and alternative embodiment is shown in which the volume V(3) shown in FIG. 26 is adjustable, in order to set the apparatus to collect a certain section of breath from the exhaled gas. For example the apparatus can be set to obtain the last 50 ml of expiratory gas except for the last 35 ml inherently left in the mouthpiece and Tee. Or for example the apparatus can be set to obtain 50 ml of gas with 100 ml of expiratory gas still behind it. Or for example the apparatus can be set to obtain a 50 ml sample from the beginning of exhalation, by increasing V(3) to 415 ml. This adjustment can be made manually, automatically or semi-automatically, or alternatively different apparatuses can be made available for each situation. The adjustment shown in FIG. 32 can optionally be performed by integrating this adjustment feature with the embodiments shown in FIGS. 29-31, in which breathing signal measurements can be used to adjust the volume. In this case a simple motor or slide mechanism is built into the expiratory limb of the apparatus, which can be battery powered.

The system described in FIGS. 19-32 can be useful for collecting and measuring end-tidal gas samples, as well as samples from other sections of the breath. It can be used for measuring for example CO in the breath, or other gases, such as H2, NO, and others. It can be used for measuring other non-gaseous substances in the breath as well as gaseous markers, and used for collecting for measurement gas sections from different portions of the expiratory cycle. The system can be applied to any type of breathing and patient interface and applied to forced breathing maneuvers or spontaneous breathing, depending on the desired test.

What is claimed is:

1. A system to measure a level of an analyte in a gas sample of exhaled breath, the system comprising:
a pump to draw a flow of gas from a patient;
a breathing detector to measure a breathing signal in the flow of gas;
a main channel from the breathing detector to the pump;
a branch channel in parallel with the main channel, wherein the branch channel connects to the main channel at both ends such that gas drawn through the branch channel can bypass a first portion of the main channel;
an analyte composition sensor fluidly connected to the branch channel;
an exhaust downstream of the pump, wherein gas drawn through the branch channel exits through the exhaust, and wherein gas drawn through the first portion of the main channel exits through the exhaust;
a processor that determines an acceptable breath based on the breathing signal and determines a location of a desired section of the acceptable breath based on the breathing signal; and
a control system to divert the desired section of breath to the channel and the analyte sensor.

2. The system of claim 1, wherein a subsection of the bypass channel is isolatable and removable so that the desired section of breath can be captured and removed from the system.

3. The system of claim 1, wherein the analyte composition sensor is positioned in a side channel of the bypass channel.

4. The system of claim 1, wherein the analyte sensor is positioned inside the channel.

5. The system of claim 1, a three-way valve on the upstream end of the bypass channel.

6. The system of claim 1, a three-way valve on the downstream end of the bypass channel, wherein the control system operates the three-way valve to divert flow through the bypass channel or through the first portion of the main channel.

7. A breath sampling apparatus comprising:
a patient interface;
an inspiratory inlet;
an expiratory outlet;
a three-way junction fluidly connected to the patient interface, the inspiratory inlet, and expiratory outlet;
an inspiratory one-way valve that allows flow from the inspiratory inlet to the three-way junction;
a first expiratory one-way valve that allows flow from the three-way junction to the expiratory outlet; and
a second expiratory one-way valve that allows flow from the three-way junction to the expiratory outlet, wherein the second expiratory one-way valve is positioned downstream of the first expiratory one-way valve.

8. The breath sampling apparatus of claim 7, further comprising a gas sample extraction port positioned between the first expiratory one-way valve and the second expiratory one-way valve.

9. The breath sampling apparatus of claim 7, further comprising a removable chamber between the three-way junction and the expiratory outlet.

10. The breath sampling apparatus of claim 7, wherein a diameter of a gas pathway of the apparatus is between 0.375 inches to 0.75 inches.

11. The breath sampling apparatus of claim 7, an adjustable section between the three-way juncture and the expiratory outlet.

12. A breath sampling apparatus comprising:
a patient interface;
an inspiratory inlet;
a three-way valve;
a three-way junction fluidly connected to the patient interface, inspiratory inlet, and the three-way valve;
an inspiratory one-way valve that allows flow from the inspiratory inlet to the three-way junction;
a first expiratory outlet;
a second expiratory outlet, wherein the three-way valve is fluidly connected to the first expiratory outlet, the second expiratory outlet, and the three-way junction;
an expiratory one-way valve that allows flow from the three-way valve to the second expiratory outlet;
a breathing sensor;
a processor that receives a signal from the breathing sensor, identifies a breath sample based on the signal, and diverts flow from the first expiratory outlet to the second expiratory outlet so that the breath sample does not flow through the first expiratory outlet.

13. The breath sampling apparatus of claim 12, further comprising a gas sample extraction port positioned between the three-way valve and the expiratory one-way valve.

14. The breath sampling apparatus of claim 12, further comprising a removable chamber between the three-way valve and the expiratory outlet.

15. The breath sampling apparatus of claim 14, wherein the removable chamber comprises the expiratory outlet.

16. The breath sampling apparatus of claim 12, wherein the breathing sensor is positioned between the inspiratory patient interface and the three-way valve.

17. The breath sampling apparatus of claim 12, further comprising a removable mouthpiece.

18. The breath sampling apparatus of claim 12, wherein a diameter of a gas pathway of the apparatus is between 0.375 inches to 0.75 inches.

19. The breath sampling apparatus of claim 12, an adjustable section between the three-way juncture and the expiratory outlet.

20. The breath sampling apparatus of claim 19, further comprising graduated markings on the adjustable section.
